(19) United States
(12) Patent Application Publication
(10) Pub. No.: US 2023/0218449 A1
(43) Pub. Date: Jul. 13, 2023

(54) DEVICE FOR MALE INCONTINENCE

(71) Applicant: Yvette Thompson, Landover, MD (US)

(72) Inventor: Yvette Monique Thompson, Landover, MD (US)

(21) Appl. No.: 18/151,423

(22) Filed: Jan. 7, 2023

Related U.S. Application Data
(60) Provisional application No. 63/297,275, filed on Jan. 7, 2022.
(60) Provisional application No. 63/297,858, filed on Jan. 10, 2022.

(51) Int. Cl. A61F 13/471

(52) U.S. Cl. CPC A61F 13/471; A61F 2013/15121

(57) ABSTRACT

A device for addressing male incontinence considered a sanitary napkin that fits over the penis and is easy to use. The male incontinence device provides discreet comfort and protection for men who experience light to moderate urinary incontinence.

### FIELD OF THE INVENTION

The device of the present invention relates to male incontinence control,

### BACKGROUND OF THE INVENTION

The present invention relates to an incontinence device, in particular a male pad featuring a device that provides ease of application and removal to protect against leakage due to incontinence or fluid from the penis after urination.

Incontinence is not just common in women. There are many men in the world that suffer from urinary incontinence (UI). This is a condition that can be found in men whom may have the inability to control the release of minor to moderate leakage after urinating.

### SUMMARY OF THE INVENTION

The purpose of the present invention is to eliminate the embarrassment of having stained underwear or trousers after urinating. The present invention is a male incontinence device that allows the user to insert the head/tip of the penis through an opening into a tubular fluid proof layer as defined in this device. The pad has a collar, a tubular body extending from the collar, and at the closed end an absorbent material affixed therein.

The collar and tubular fluid proof layer provide snugness for which the fit limits the leakage. Studies indicate that men between the ages of 40 and 70 experience this problem. I recognize the need for this device and feel it is a sleek way of disguising the problem of fluid leakage for men.

A study of a male subject shows that after ten days of wearing the device of the present invention at different times using small, medium, large and extra-large cotton balls placed inside of the latex to determine the amount of fluid absorbency after urination, it was determined that the subject experienced amounts that would absorb all sizes of the cotton depending upon what he consumed, i.e., sometimes the male subject would soak the extra-large cotton ball and sometimes only enough would be there for a small cotton ball.

Most incontinence pads in the United States for men are similar to a woman’s sanitary napkin. The male incontinence pad in comparison to the women’s sanitary napkin is affixed to the inside of the boxer or brief. This product does not solve the problem of incontinence for those males who do not like to wear underwear or feel as if a pad in their underwear or trouser is too bulky or uncomfortable.

The present invention not only provides a snug fit but has a smooth texture with a skin conditioning emollient or lubricant to prevent chaffing of the head/tip of the penis and an absorbent material at the closed end that may contain a substance such as a hydrogel which transforms the fluid into a solid. The hydrogel foam material, accordingly is formed of cross linked water absorbent polymer foams containing units derived from monoethylenically unsaturated acids, such as acrylic acid. The skin conditioning agent may comprise one or more of the following substances, emollients, pH regulating substances, antimicrobial substances, glucocorthoids, antiviral agents, enzyme inhibitors and anti-inflammatory substances.

The device of the present invention is fitted about the head of the penis after urination using the fingers to secure it in place causing a vacuum. After the excess fluid is released than the device can be immediately disposed of or discarded at the next bathroom visit. Due to the absorption material being made of a product consisting of latex and cotton containing a substance such as hydrogel, it is not recommended that the device be flushed but disposed of properly in a waste receptacle.

### BRIEF DESCRIPTION OF THE DRAWINGS

FIG. 1 is a perspective view of a male incontinence device equipped with a collar and elongated stretchy fluid proof layer with an absorption material at the closed end of the present invention shown in the configuration as placed on the head/end of a penis, according to an embodiment.

FIG. 2 is a side elevation view of the male incontinence device wherein the device includes a collar at the base of the penis head to the closed end, according to an embodiment.

FIG. 3 is a perspective view of the male incontinence device wherein the closed end of the device has an absorption material which may consist of cotton, according to an embodiment.

### DETAILED DESCRIPTION OF THE PREFERRED EMBODIMENTS

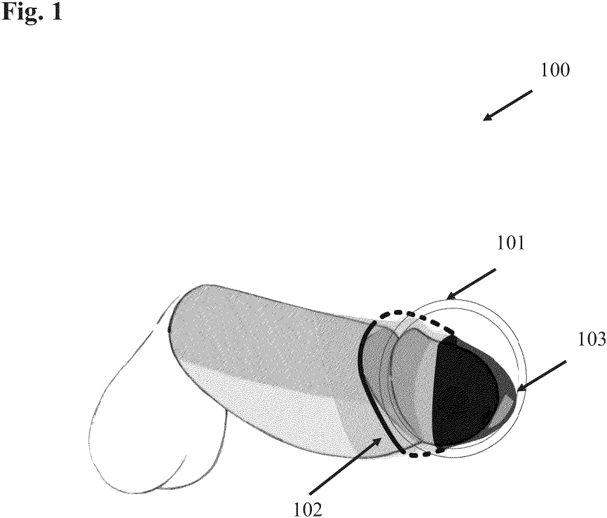

The incontinence device as described in FIG. 1 describes a product placed on the user’s skin to absorb and contain fluid from a penis 100. The invention relates to a device that is disposable and not intended to be reused. The collar as described at 100 shows at 101 a collar and a fluid proof tubular component at 102 and an absorbent material located 103. In an embodiment, the absorbent material may include cotton. The male incontinence device 100 has three principle components: a collar ring 101, a fluid proof tubular component 102 and an end portion 103 that provides a reservoir for collecting the fluid or urine.

A proximal base end 103 of the male incontinence device 102 borders the collar at 101. The collar 101 is attached to the head/end of the penis which the fluid proof tubular sheath 102 keeps the fluids incased so as to be absorbed by the absorbent material at 103.

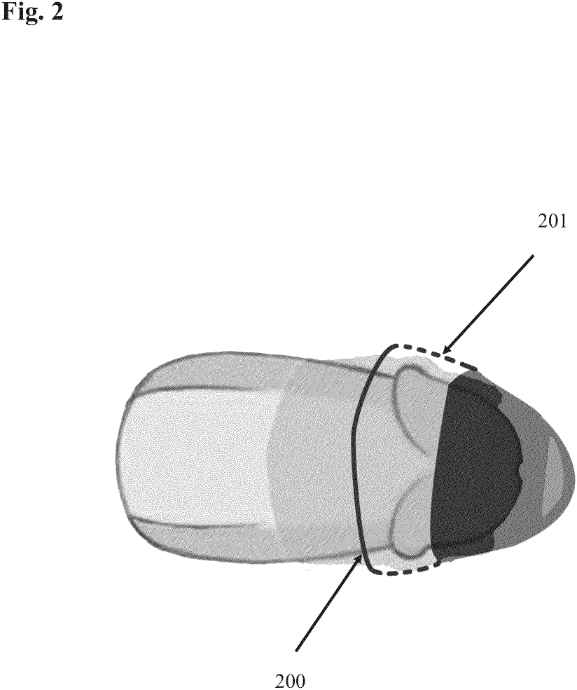

According to an embodiment the collar and sheath at 101 and 102 is made of a soft, comfortable and durable material such as biodegradable latex, lambskin or polyurethane, fabricated as shown in FIG. 2.

The fluid proof tubular component as described in FIG. 2 at 200 may accommodate varying penis head sizes. The device of the present invention can be made in varying sizes to accommodate the varying penis head sizes. In accordance with 201 the fluid proof tubular sheath has an opening for receiving the head of the penis; the collar is fitted thereon and extends down to the tip of the penis at 300.

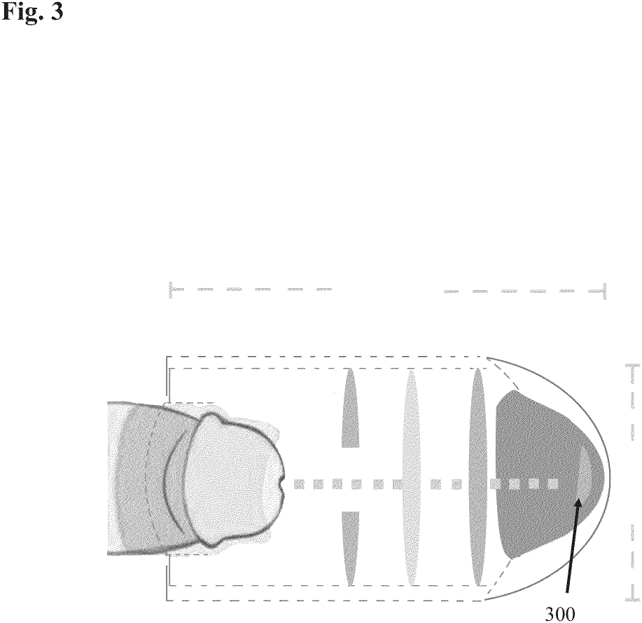

The embodiment in FIG. 3 comprises an inner soft fibrous material 300 that may consist of a substance such as cotton. The material 300 has an inner liquid-permeable material that turns the fluid to solid or gel.

Once in place the male incontinence device 100 can be easily and comfortable adjusted in the trousers or freely without clothing allowing for a custom fit for every man every time enabling him to sustain his incontinence privately.

The disposable male incontinence device 100 can be used with any commercially available lubricants designed for moisture and is completely smooth and comfortable and will not cause any chaffing.

What is claimed is:

1. A male incontinence device comprising:
a collar;
an outer stretchy fluid proof layer having a smooth texture; wherein the fluid proof layer is tubular, closed at one end and extends from the collar;
an absorption material inserted at the bottom of the fluid proof layer;
a lubricant applied to the inside of the fluid proof layer.

2. The device according to claim 1 wherein a circumference of the device is one of three or more sizes.

3. The device according to claim 1 wherein the collar and fluid proof layer are made of latex.

4. The device according to claim 1 wherein the absorption material comprises cotton.

5. The device according to claim 1 wherein the absorption material comprises a hydrogel foam.

6. The device according to claim 1 wherein the device is disposable.
